(19) United States
(12) Patent Application Publication
(10) Pub. No.: US 2013/0098373 A1
(43) Pub. Date: Apr. 25, 2013

(54) ORAL APPLIANCE AND METHODS

(71) Applicant: Intellectual Property Holdings, LLC, Cleveland, OH (US)

(72) Inventor: Nick Carlone, Highland Heights, OH (US)

(73) Assignee: INTELLECTUAL PROPERTY HOLDINGS, LLC, Cleveland, OH (US)

(21) Appl. No.: 13/660,251

(22) Filed: Oct. 25, 2012

Related U.S. Application Data
(60) Provisional application No. 61/551,141, filed on Oct. 25, 2011.
(60) Provisional application No. 61/551,268, filed on Oct. 25, 2011.
(60) Provisional application No. 61/662,446, filed on Jun. 21, 2012.

(51) Int. Cl. A61F 5/56

(52) U.S. Cl. CPC A61F 5/566
USPC 128/848; 249/187.1; 264/16

(57) ABSTRACT

The present application discloses an oral appliance, a device for making an oral appliance, a method of making an oral appliance, and a method of using an oral appliance. In certain embodiments, the oral appliance comprises first, second, and third moldable parts. Each of the first and second moldable parts is configured to form an impression of one or more of a user's upper teeth and the third moldable part is configured to form an impression of one or more of the user's lower teeth. The first and second moldable parts are coupled to the third moldable part to form the oral appliance and are adjustable relative to the third moldable part. The moldable parts facilitate positioning the user's jaw such that there is vertical separation between the maxilla and the mandible when the oral appliance is installed in the user's mouth.

### CROSS REFERENCE TO RELATED APPLICATIONS

This application is a U.S. Non-Provisional Patent Application which claims priority to U.S. Provisional Patent Application No. 61/551,268, filed on Oct. 25, 2011 and titled “Oral Appliance,” U.S. Provisional Patent Application No. 61/551,141, filed on Oct. 25, 2011 and titled “Sleep Monitoring System,” and U.S. Provisional Patent Application No. 61/662,446, filed on Jun. 21, 2012 and titled “Oral Appliance,” all of which are hereby incorporated by reference in their entirety.

### BACKGROUND

Obstructive sleep apnea (“OSA”) or obstructive sleep apnea syndrome is the most common type of sleep apnea and is caused by obstruction of the upper respiratory tract. OSA is characterized by repetitive pauses in breathing during sleep, despite the effort to breathe, and is usually associated with a reduction in blood oxygen saturation. There are a variety of treatments for OSA, including positive airway pressure therapy, invasive surgery, and oral appliance therapy (“OAT”). OAT is an effective way to treat mild to moderate OSA. OAT can, however, be expensive due in part to the cost of the OAT device. Further, OAT may not always be effective for certain patients.

### SUMMARY

The present application discloses an inexpensive and adjustable oral appliance, a device for making an oral appliance, a method of making an oral appliance, and a method of using an oral appliance. The oral appliance may be used to test patients for OSA and the efficacy of OAT. The oral appliance may also be used as a permanent device to treat mild to moderate forms of OSA.

In certain embodiments, the oral appliance comprises first, second, and third moldable parts. Each of the first and second moldable parts is configured to form an impression of one or more of a user's upper posterior teeth. Further, each of the first and second moldable parts comprise a front portion, a rear portion, and a top surface sloping downward from the front portion to the rear portion. The third moldable part is configured to form an impression of one or more of the user's lower teeth. The first and second moldable parts are coupled to the third moldable part to form the oral appliance and are adjustable relative to the third moldable part. The top surfaces of the first and second moldable parts facilitate positioning the user's jaw such that there is vertical separation between the maxilla and the mandible when the oral appliance is installed in the user's mouth.

In certain embodiments, the device for making an oral appliance comprises first, second, and third moldable parts and a frame for molding the moldable parts. Each of the first and second moldable parts is configured to form an impression of one or more of a user's upper teeth and the third moldable part is configured to form an impression of one or more of the user's lower teeth. The frame comprises a first upper channel, a second upper channel, and one or more lower channels. The first moldable part is at least partially disposed within the first upper channel, the second moldable part is at least partially disposed within the second upper channel, and the third moldable part is at least partially disposed within the one or more lower channels.

In certain embodiments, the method of making an oral appliance comprises placing a molding device in boiling water. The molding device comprises a first moldable part, a second moldable part, a third moldable part, and a frame. The first and second moldable parts are configured to form an impression of one or more of a user's upper teeth and the third moldable part is configured to form an impression of one or more of the user's lower teeth. The moldable parts are disposed within channels of the frame. The molding device is removed from the boiling water and inserted in a user's mouth. The user bites down on the moldable parts to form an impression of the user's teeth in the moldable parts. The molding device is removed from the user's mouth and the moldable parts are removed from the frame of the molding device. The first and second moldable parts are coupled to the third moldable part to form the oral appliance.

These and additional embodiments will become apparent in the course of the following detailed description. The descriptions of the embodiments below are not intended to and do not limit the scope of the words of the claims in any way. The words of the claims have all of their full, ordinary meanings.

### BRIEF DESCRIPTION OF THE DRAWINGS

FIG. 1A is an exploded top front perspective view of a molding device according to an embodiment of the present application.

FIG. 1B is a top front perspective view of the molding device shown in FIG. 1A.

FIG. 1C is a partial rear view of the molding device shown in FIG. 1A taken along line 1C-1C of FIG. 1B.

FIGS. 2A-2E are top front perspective, rear, top, bottom, and right side views, respectively, of a frame of a molding device according to an embodiment of the present application.

FIG. 3 is a top front perspective view of a frame of a molding device according to an embodiment of the present application.

FIG. 4A is an exploded top front perspective view of an upper moldable part according to an embodiment of the present application.

FIGS. 4B-4D are left side, top, and bottom views, respectively, of the upper moldable part shown in FIG. 4A.

FIG. 4E is a side cross sectional view of the upper moldable part shown in FIG. 4A taken along line 4E-4E of FIG. 4C.

FIG. 5A is an exploded top front perspective view of a lower moldable part according to an embodiment of the present application.

FIGS. 5B and 5C are top and left side views of the lower moldable part shown in FIG. 5A.

FIG. 6 is a top front perspective view of a base portion of a lower moldable part according to an embodiment of the present application.

FIG. 7A is a cross sectional rear view of the upper moldable part shown in FIG. 4A and the frame shown in FIG. 2A taken along line 7A-7A of FIG. 4C and line 7A-7A of FIG. 2C, respectively.

FIG. 7B is a cross sectional rear view of the lower moldable part shown in FIG. 5A and the frame shown in FIG. 2A taken along line 7B-7B of FIG. 5B and line 7B-7B of FIG. 2C, respectively.

FIG. 8A is an exploded top front perspective view of an oral appliance according to an embodiment of the present application.

FIGS. 8B and 8C are top front perspective and top views of the oral appliance shown in FIG. 8A.

FIGS. 9A-9E are cross sectional side views of the oral appliance shown in FIG. 8A taken along line 9C-9C of FIG. 8C and showing an upper moldable part adjusted at various positions relative to a lower moldable part of the oral appliance.

FIG. 10A is an exploded top front perspective view of a molding device according to an embodiment of the present application.

FIGS. 10B and 10C are top front perspective and bottom rear perspective views of the molding device shown in FIG. 10A.

FIG. 11 is a side view of an oral appliance according to an embodiment of the present application.

FIGS. 12A and 12B illustrate downward and forward movement of a person's jaw.

### DESCRIPTION OF EMBODIMENTS

The oral appliance of the present application may be used as a temporary OAT device that permits patients to be tested for OSA and the efficacy of OAT, e.g., before having to incur the cost of a more permanent OAT device. There are various methods for an individual to be tested for OSA. Two of the most common methods are for the patient to spend a night in a sleep lab or undergo a Home Sleep Test (“HST”). The oral appliance of the present application permits these tests to show the effectiveness of OAT for an individual who undergoes a study. For example, the patient may be tested with and without the oral appliance to assess whether OAT may be an effective treatment for OSA. The oral appliance of the present application may also be used as a more permanent device to treat mild to moderate forms of OSA.

In certain embodiments, the oral appliance is formed by heating one or more moldable parts in hot or boiling water and then placing the heated moldable parts in the patient's mouth to form an impression of the upper and lower teeth. The moldable parts are then assembled to form the oral appliance. In this regard, the oral appliance can be made and assembled in a short period of time (e.g., in about 2 to about 5 minutes or less) and by a patient in their own home.

The molding device for making an oral appliance of the present application generally comprises a plurality of moldable parts and a frame for molding the moldable parts. The moldable parts are configured to form an impression of one or more of a user's upper and lower teeth and the frame comprises a plurality of channels. The moldable parts are at least partially disposed within the channels of the frame. Once an impression of the user's upper and lower teeth are formed in the moldable parts, the moldable parts removed from the frame and are coupled together to form the oral appliance.

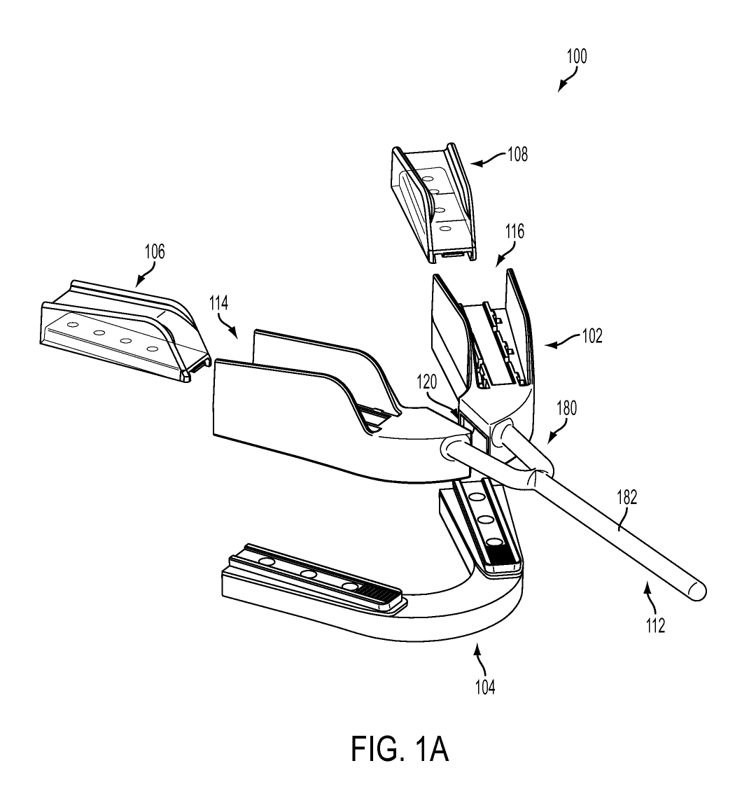

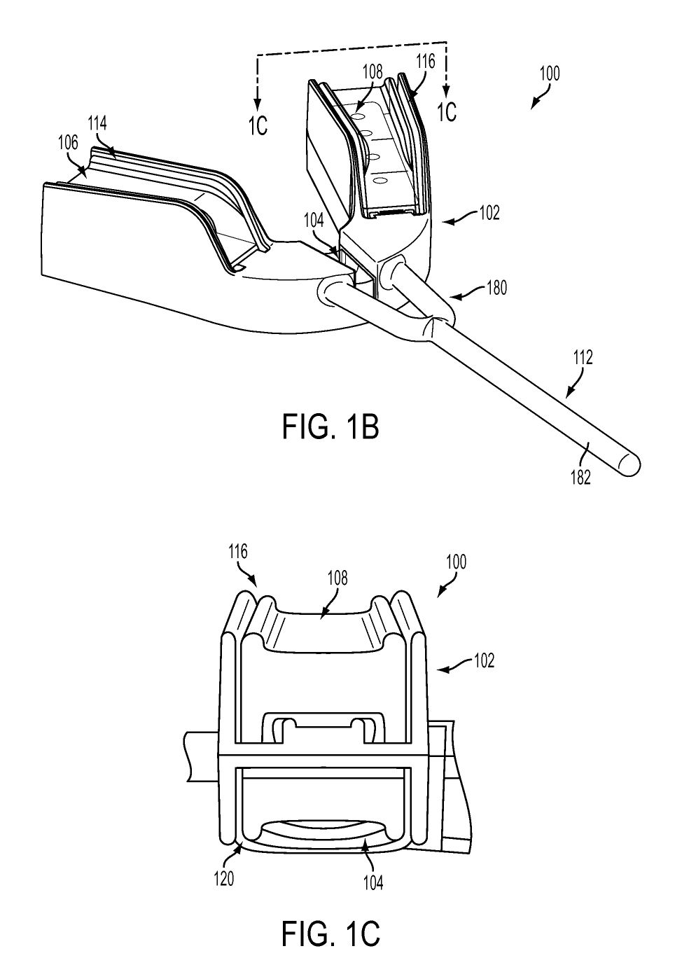

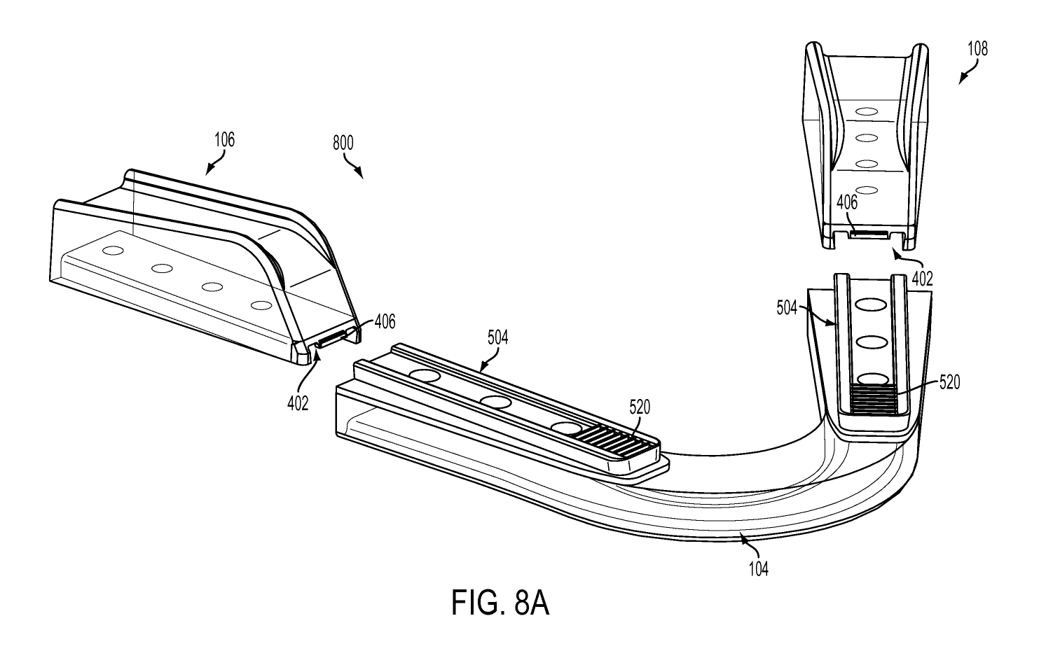

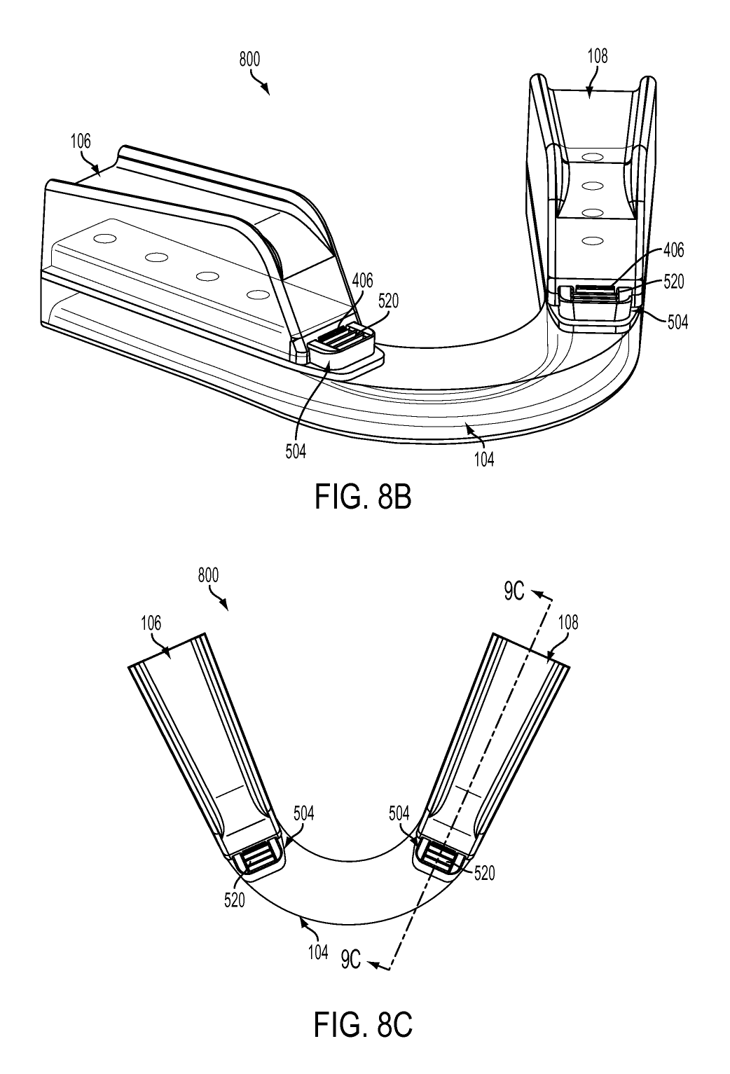

FIGS. 1A-1C illustrate a molding device 100 for making an oral appliance according to an embodiment of the present application. As shown, the molding device 100 is U-shaped and configured to fit a human mouth. The molding device 100 comprises a frame having a handle or holder 112 and a plurality of moldable parts. The moldable parts include a first moldable part or right upper portion 106, a second moldable part or left upper portion 108, and a third moldable part or lower portion 104. Once molded, the first and second moldable parts 106 and 108 are adjustably coupled to the third moldable part 104 to form an oral appliance 800 shown in FIGS. 8A-8C.

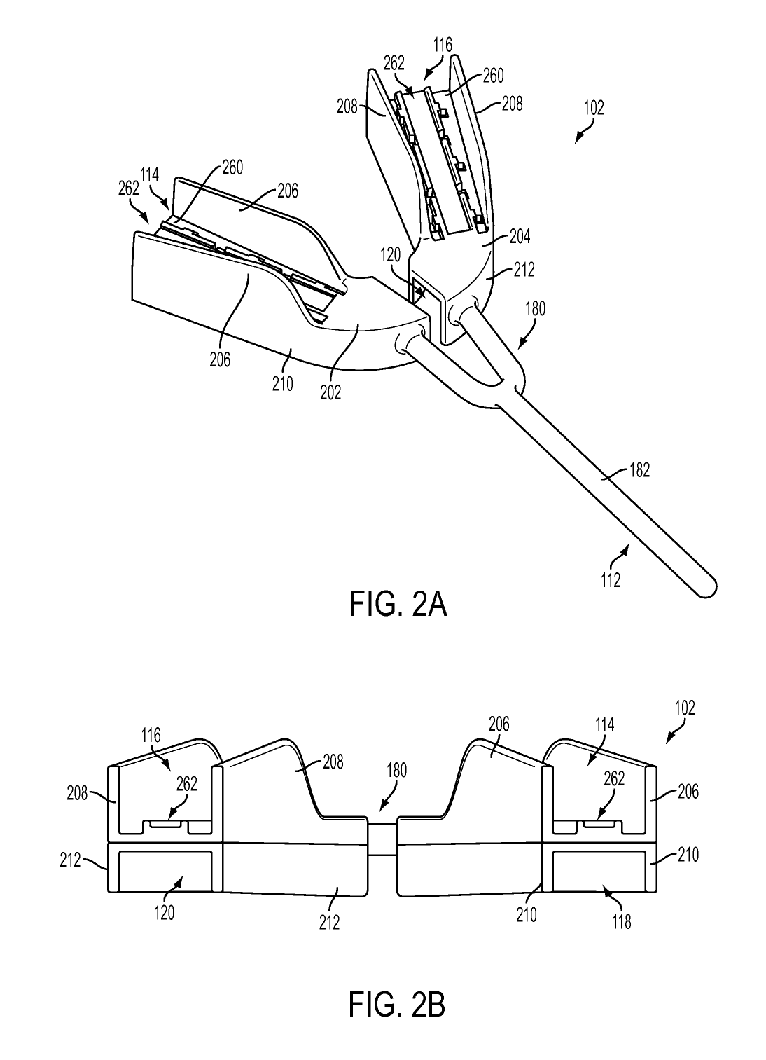

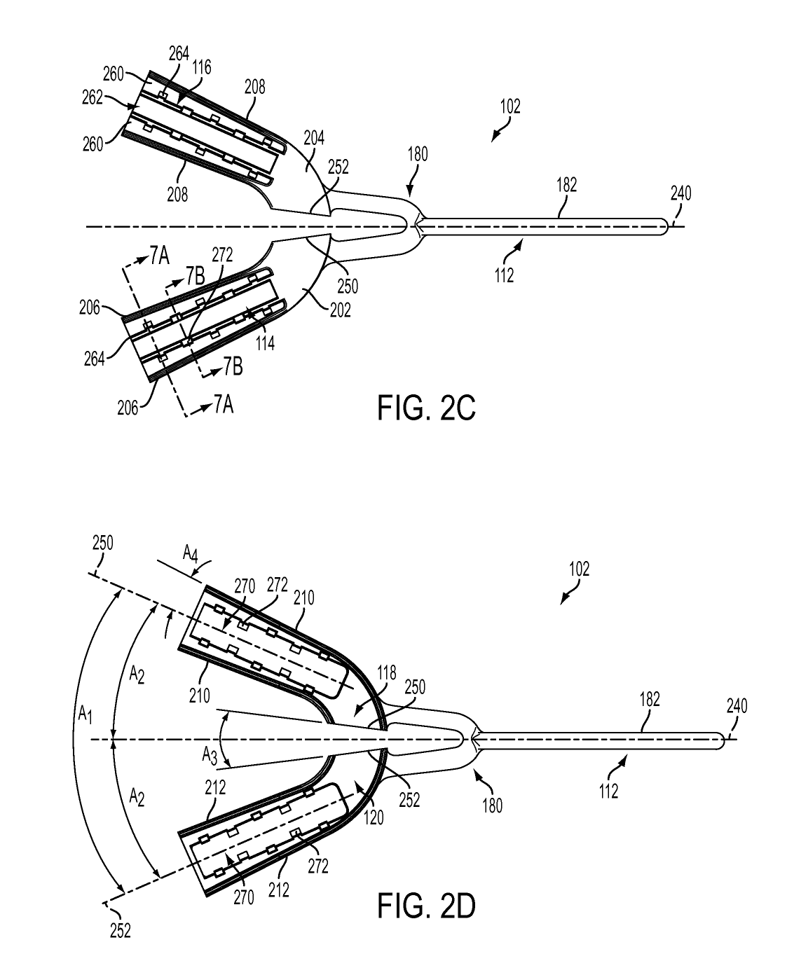

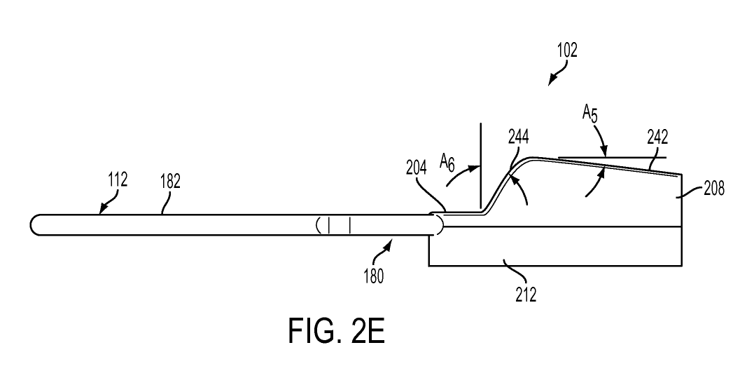

FIGS. 2A-2E illustrates an embodiment of a frame 102 of the molding device 100. The frame 102 acts as the form or mold for the first and second moldable parts 106 and 108 and the third moldable part 104. The frame 102 comprises a first upper channel 114, a second upper channel 116, a first lower channel 118, and a second lower channel 120. As illustrated in FIGS. 2A-2D, the frame 102 is divided into separate right and left portions, or first and second portions, that are symmetrical about a centerline 240 of the frame. Dividing the frame 102 in this manner permits the right and left portions to flex and move relative to each other and facilitates insertion, attachment, and removal of the third moldable part 104 from the lower channels 118 and 120 of the frame. Further, the right and left portions may be moved relative to each other (e.g., towards or away from one another) to fit the size, shape, and configuration of the patient's mouth or teeth. For example, the right and left portions may be moved towards one another to fit a smaller mouth and away from one another to fit a larger mouth.

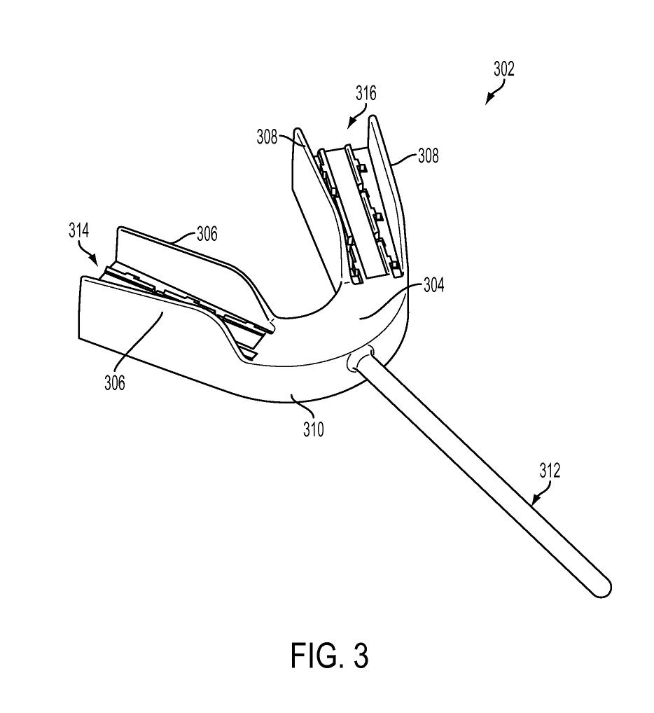

In certain embodiments, the frame of the present application may not be divided into separate right and left portions and/or may comprise a continuous upper channel and/or a continuous lower channel. For example, FIG. 3 illustrates a frame 302 according to an embodiment of the present application. As shown, the frame 302 comprises a first upper channel 314, a second upper channel 316, and a continuous lower channel. The frame 302 may also be used to form or mold the first and second moldable parts 106 and 108 and the third moldable part 104.

The upper and lower channels of the frame are configured to receive the moldable parts and facilitate molding of the moldable parts by prohibiting the movement and/or outward deformation of the moldable parts during the molding process as the impression of the upper and lower teeth is formed. As illustrated in FIGS. 1A-1C, the first moldable part 106 is at least partially disposed within the first upper channel 114, the second moldable part 108 is at least partially disposed within the second upper channel 116, and the third moldable part 104 is at least partially disposed within the first and second lower channels 118 and 120 of the frame 102. Further, the frame 302 is configured such that the first moldable part 106 is at least partially disposed within the first upper channel 314, the second moldable part 108 is at least partially disposed within the second upper channel 316, and the third moldable part 104 is at least partially disposed within the lower channel.

The frame of the present application may comprise one or more center portions, a plurality of sidewalls extending upward from the one or more center portions to form the first and second upper channels, and a plurality of sidewalls extending downward from the one or more center portions to form the one or more lower channels. As illustrated in FIGS. 2A-2E, the frame 102 comprises a first center portion 202, a second center portion 204, first upper sidewalls 206 extending upward from the first center portion to at least partially form the first upper channel 114, first lower sidewalls 210 extending downward from the first center portion to at least partially form the first lower channel 118, second upper sidewalls 208 extending upward from the second center portion to at least partially form the second upper channel 116, and second lower sidewalls 212 extending downward from the second center portion to at least partially form the second lower channel 120. As illustrated in FIG. 3, the frame 302 comprises a center portion 304, first upper sidewalls 306 extending upward from the center portion to at least partially form the first upper channel 314, second upper sidewalls 308 extending upward from the center portion to at least partially form the second upper channel 316, and lower sidewalls 310 extending downward from the center portion to at least partially form the lower channel.

The upper channels of the frame are configured to at least partially surround a portion of the upper posterior teeth (i.e., the molars and bicuspids) of the patient. As illustrated in FIGS. 1A-1C, the first and second moldable parts 106 and 108 are disposed within the upper channels 114 and 116 of the frame 102 and at least partially fill the upper channels. The shape of the upper channels 114 and 116 follow the shape of the first and second moldable parts 106 and 108 to facilitate molding of these parts. Further, the shape of the upper channels 314 and 316 of the frame 302 are configured to follow the shape of the first and second moldable parts 106 and 108 to facilitate molding of these parts.

The one or more lower channels of the frame are configured to at least partially surround the lower teeth of the patient. As illustrated in FIGS. 1A-1C, the third moldable part 104 is disposed within the lower channels 118 and 120 and at least partially fills the lower channels. The shape of the lower channels 118 and 120 follow the shape of the third moldable part 104 to facilitate molding of this part. Further, the shape of the lower channel of the frame 302 is configured to follow the shape of the third moldable part 104 to facilitate molding of this part.

The first and second upper channels of the frame comprise a front portion and a rear portion and the depth of the upper channels may vary between the front portion and the rear portion of the channel. For example, as illustrated in FIGS. 2A, 2B, and 2E, the first and second upper channels 114 and 116 of the frame 102 have a varying depth. The top edge of each upper sidewall 206 and 208 slopes downward from a front or leading edge of the sidewall to a rear edge of the sidewall such that the upper channels 114 and 116 are deeper towards the front portion of the channel than the rear portion of the channel. As such, the upper channels 114 and 116 are deeper towards the front portion of the upper posterior teeth than the rear portion of the upper posterior teeth. Similarly, as illustrated in FIG. 3, the top edge of each upper sidewall 306 and 308 of the frame 302 slopes downward from a front or leading edge of the sidewall to a rear edge of the sidewall such that the upper channels 314 and 316 are deeper towards the front portion of the channel than the rear portion of the channel.

For example, as illustrated in FIG. 2E, the angle A₅ of the sloping top edge 242 of the upper sidewalls 206 and 208 may be between 0 degrees (no slope) and about 10 degrees relative to horizontal. In certain embodiments, the depth of the upper channels 114 and 116 may be no greater than about 0.5 inch at the front of the channel and no greater than about 0.3 inch at the rear of the channel. Further, the front or leading edge 244 of the upper sidewalls 206 and 208 extends rearward from the center portions 202 and 204 to the top edge 242 at an angle A₆ between 0 degrees and about 45 degrees relative to vertical. In certain embodiments of the molding device 100, the angle A₅ of the sloping top edge 242 of each upper sidewall 206 and 208 is about 8 degrees relative to horizontal and the angle A₆ of the front edge 244 is about 30 degrees relative to vertical.

Further, the depth of the one or more lower channels may vary. For example, the lower channels 118 and 120 may be slightly deeper towards a front portion of the channel than a rear portion of the channel. As such, the lower channels 118 and 120 may be slightly deeper around the lower anterior teeth than the lower posterior teeth. For example, in certain embodiments the depth of the lower channels 118 and 120 may be no greater than about 0.4 inch at the anterior or front portion of the channel and no greater than about 0.3 inch at the posterior or rear portion of the channel. Further, the lower channel of the frame 302 may be slightly deeper towards the front portion of the channel than the rear portions of the channel such that the lower channel is slightly deeper around the lower anterior teeth than the lower posterior teeth.

The frame of the present application is generally U-shaped and configured to fit in a human mouth. As illustrated in FIG. 2D, the centerlines 250 and 252 of the right and left portions of the frame 102 extend at angle A₂ relative to the centerline 240 of the frame and form an angle Aₗ between the centerlines of the right and left portions. The angle A₂ may vary between about 0 degrees and about 50 degrees and the angle A₁ may vary between about 0 degrees and about 100 degrees. In certain embodiments, the angle A₂ is about 24 degrees and the angle Aₗ is about 48 degrees when the frame 102 is in a neutral position. However, as discussed above, the right and left portions of the frame 102 may be flexed and moved relative to each other to increase or decrease the angle A₂ and/or the angle A₁.

As illustrated in FIG. 2D, the ends 250 and 252 of the lower channels 118 and 120 and center portions 202 and 204 are angled relative to the centerline 240 of the frame. An angle A₃ between the ends 250 and 252 may vary between about 0 degrees and about 100 degrees. In certain embodiments, the angle A₃ is about 15 degrees when the frame 102 is in a neutral position. However, as discussed above, the right and left portions of the frame 102 may be flexed and/or moved relative to each other to increase or decrease the angle A₃.

The upper and lower sidewalls of the frame may flare outward relative to the centerline of the channel from the front portion to the rear portion of the channel. As such, the rear portion of the channel may be wider than the front portion of the channel. For example, as illustrated in FIG. 2D, the upper sidewalls 206 and 208 and the lower sidewalls 210 and 212 flare outward relative to the centerlines 250 and 252 to form an angle A₄ between the centerlines and the sidewalls. The angle A₄ may vary between about 0 degrees and about 100 degrees. In certain embodiments, the angle A₄ is about 3 degrees.

The upper and lower channels of the frame may also include features that are configured to mate with corresponding features on the moldable parts received in the channel. The features on the channel and/or the moldable part may include, for example, guides, tracks or rails, openings, bosses, protrusions, barbs, notches, grooves, ridges, slots, or the like of various shapes and sizes. The features may be configured to facilitate alignment of the moldable part in the channel and prohibit movement of the moldable part as the impression of the upper and lower teeth is formed.

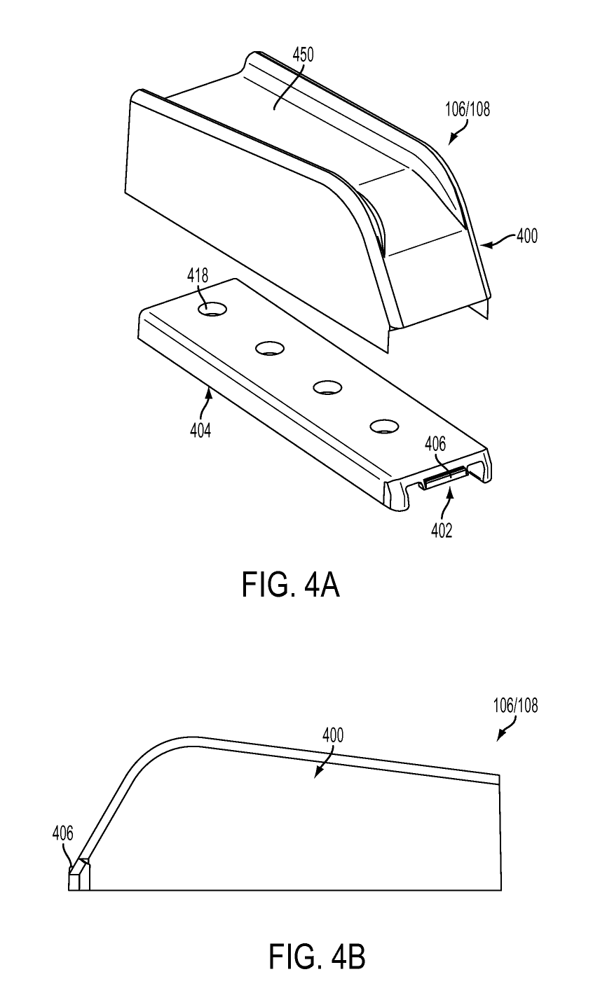

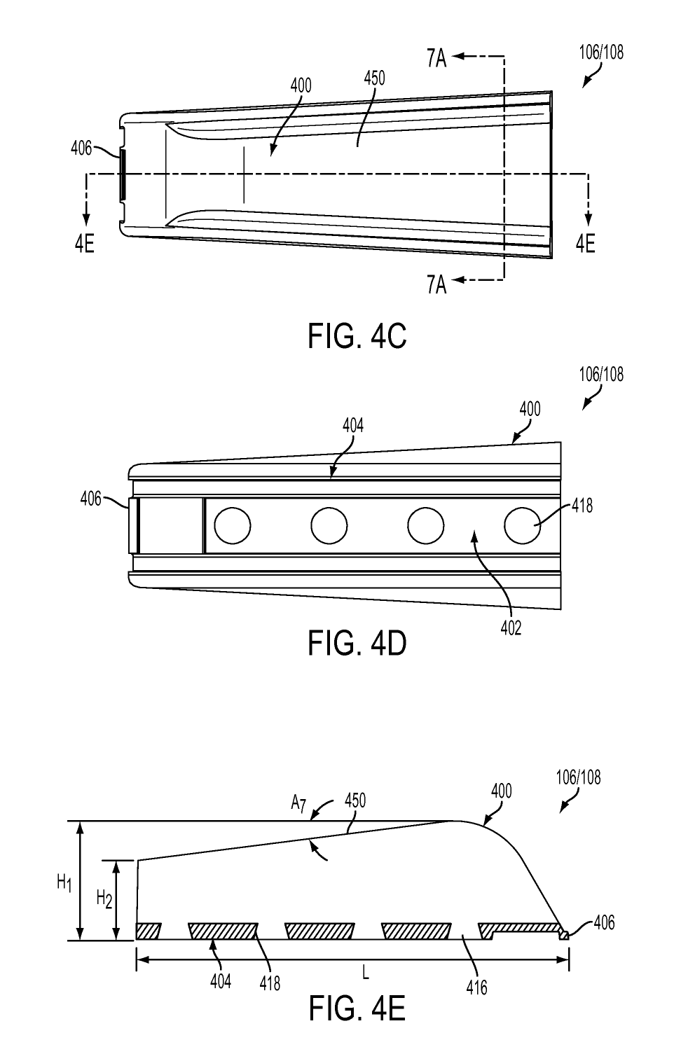

As illustrated in FIGS. 2A and 2C, the center portions 202 and 204 of the frame 102 include grooves 260 in the upper channels 114 and 116 that form a track portion 262 for guiding the first and second moldable parts 106 and 108. The track portion 262 is configured for receipt of a guide portion 402 (FIG. 4A) of the first and second moldable parts 106 and 108 to facilitate alignment of those parts in the upper channels 114 and 116. The track portion 262 and the grooves 260 of the center portions 202 and 204 also prohibit the movement of the first and second moldable parts 106 and 108 out of the upper channels 114 and 116.

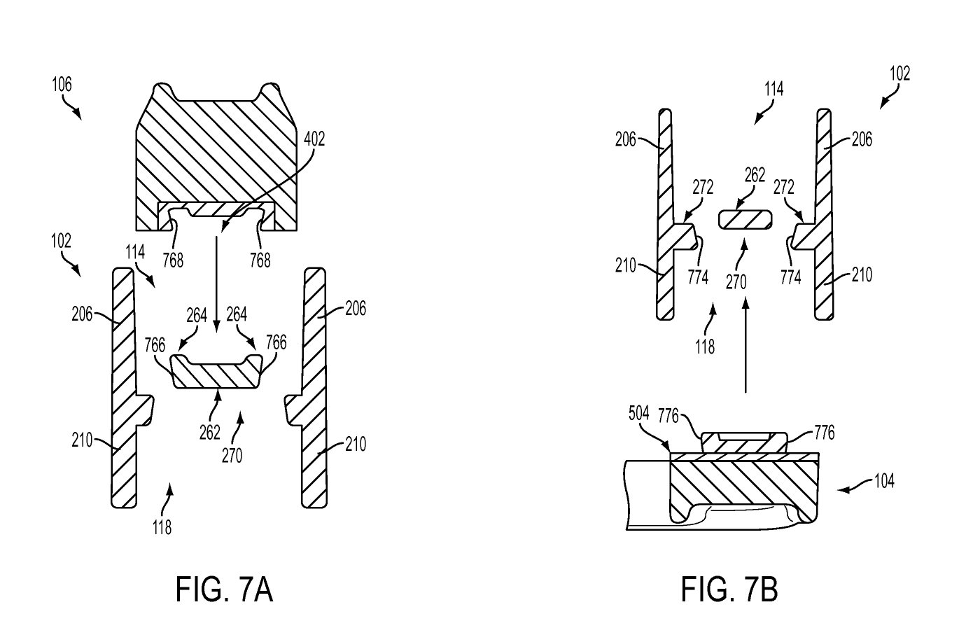

As illustrated in FIGS. 2C and 7A, the track portion 262 comprises a plurality of protrusions 264 spaced along the length of the track portion and extending outward from the track portion that prohibit movement of the first and second moldable parts 106 and 108 out of the upper channels 114 and 116. As shown in FIG. 7A, the protrusions 264 comprise angled surfaces 766 that abut corresponding angled surfaces 768 of the guide portion 402 when the first and second moldable parts 106 and 108 are received within the upper channels 114 and 116. Further, the grooves 260 in the center portions 202 and 204 of the frame 102 are sized and shaped such that the front of the guide portion 402 abuts the forward portion of the grooves when the first and second moldable parts 106 and 108 are received in the upper channels 114 and 116. As illustrated in FIGS. 2A and 2C, the track portion 262 also comprises a recessed top surface that provides clearance for a lip or catch 406 (FIG. 4A) of the first and second moldable parts 106 and 108 when they are slid along the track portion and into the upper channels 114 and 116.

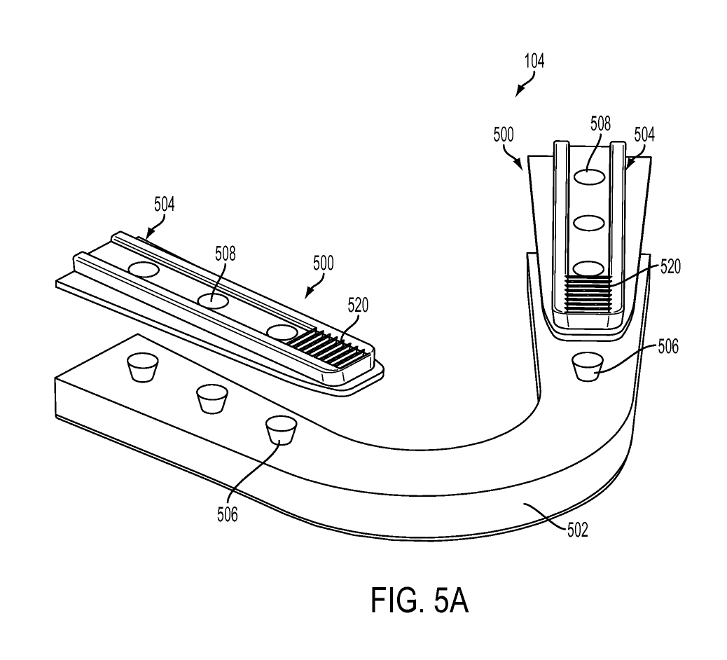

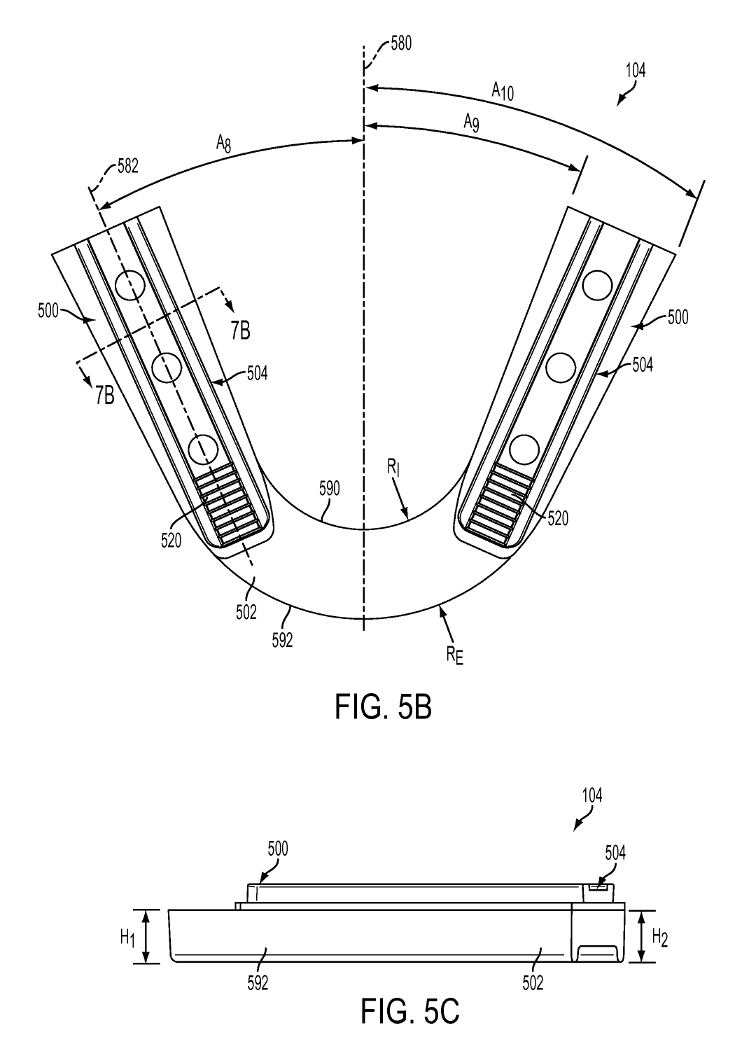

As illustrated in FIG. 2D and 7B, the center portions 202 and 204 of the frame 102 include recessed portions 270 in the lower channels 118 and 120. The recessed portions 270 are configured to receive a track portion 504 (FIG. 5A) of the third moldable part 104 to facilitate alignment of the part and prohibit movement of the part relative to the lower channels 118 and 120. As illustrated in FIGS. 2D and 7B, the recessed portions 270 comprise a plurality of protrusions 272 spaced along the length of the recessed portion and extending inward that prohibit movement of the third moldable part 104 out of the lower channels 118 and 120. As shown in FIG. 7B, the protrusions 272 comprise angled surfaces 774 that abut corresponding angled surfaces 776 of the track portion 504 when the third moldable part 104 is received within the lower channels 118 and 120.

Furthermore, the first, second, and third moldable parts are shaped and configured to be frictionally held in the upper and lower channels of the frame 102. In this regard, a friction or interference fit is formed between the sidewalls of the upper and lower channels and the moldable parts to hold the moldable parts in the channels as the impression of the upper and lower teeth is formed.

As illustrated in FIGS. 1A-2E, the frame 102 comprises an attachment portion 180 that connects the right and left portions of the frame together and permits the right and left portions to flex and move relative to one another. As shown, the attachment portion 180 is configured as a wishbone shaped member having a first end attached to the right portion and a second end attached to the left portion. However, the right and left portions of the frame 102 may be attached in a variety of other ways that permit movement of the right and left portions relative to one another, such as, for example, with a flexible or elastic member, a living hinge, or various types of connectors. Extending from the attachment portion 180 is an elongated member 182 of the holder 112. The elongated member 182 permits the user to be positioned away from the hot or boiling water during the molding or forming process. As illustrated in FIG. 3, the holder 312 of the frame 302 comprises an elongated member extending from the lower sidewall 310. The attachment portion and/or the holder of the present application made from a single piece of material or may comprise multiple pieces or components attached together or integrally formed to produce the attachment portion and/or holder.

The frame of the present application may be made from a single piece of material or multiple pieces or components attached together or integrally formed to produce the frame. The frame may also be made of a variety of materials that may be used in the mouth. Further, the frame may be made of a different material than the moldable parts to facilitate removal of the moldable parts from the frame after molding. For example, in one embodiment, the frame is molded and made of a thermoplastic polymer such as polypropylene. This material facilitates removal of the frame from the moldable parts after molding is completed. In another embodiment, the frame is made of an ethylene vinyl acetate (EVA) copolymer. One example of this material is Ateva® 2803G having a 28% vinyl acetate content; a melt index of 3.0 g/10 min; a density of 952 kg/m²; a vicat softening point of 44° C.; and a hardness of 81 (Shore A) and 28 (Shore D). However, a variety of other materials may be used.

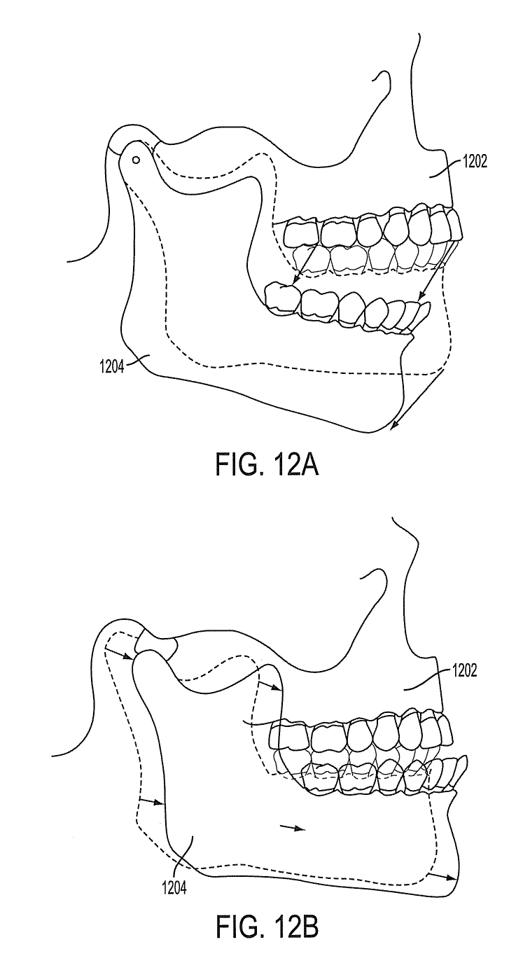

Once molded, the moldable parts are removed from the frame and the first and second moldable parts 106 and 108 are removably coupled to the third moldable part 104 to form the oral appliance 800 shown in FIGS. 8A-8C. When positioned in the patient's mouth, the oral appliance 800 is configured to maintain the mandible of the patient in an open and forward position relative to the normal posture of the jaw during sleep to reduce snoring and/or apneic episodes caused by OSA. As discussed below, the moldable parts are configured such that the mandible is moved downward (e.g., hinge movement of the mandible) relative to the maxilla when the oral appliance 800 is positioned or installed in the patient's mouth. As such, there is vertical separation between the mandible and maxilla of the patient and the mandible or jaw of the patient is in an open position. For example, FIG. 12A illustrates downward movement of the mandible 1204 relative to the maxilla 1202 such that there is vertical separation between the mandible and maxilla and the mandible or jaw is in an open position. The moldable parts are also configured such that the mandible is moved forward (e.g., translatory movement of the mandible to a anterior, protruded position) relative to the maxilla when the oral appliance 800 is positioned or installed in the patient's mouth such that the mandible is in a forward position. For example, FIG. 12B illustrates forward movement of the mandible 1204 relative to the maxilla 1202 such that the mandible is in a forward position. As discussed below, the first and second moldable parts 106 and 108 may also be adjusted relative to the third moldable part 104 to move the mandible more or less forward relative to the maxilla of the patient. Positioning the jaw of the patient in an open and forward position helps to keep the airway of the patient open during sleep.

As illustrated in FIGS. 4A-4E, the first and second moldable parts 106 and 108 include a base portion 404 and a moldable portion 400. As illustrated in FIG. 4E, the moldable portion 400 includes a plurality of spaced protrusions or bosses 416 (i.e., male portion) extending downward from a bottom surface of the moldable portion. The spaced protrusions 416 are configured to mate with a plurality of spaced openings 418 (i.e., female portion) in the base portion 404 (or vice versa). These protrusions 416 and openings 418 permit the moldable portion 400 to be removably coupled to the base portion 404. However, the base portion 404 and the moldable portion 400 may be made of a single piece of material or a plurality of materials attached together or integrally formed to form the first and second moldable parts 106 and 106. Furthermore, the base portion 404 and the moldable portion 400 may be attached together or integrally formed in a variety of ways, such as, for example, laminated, overmolded, or with one or more fasteners. As described in greater detail below, the base portion 404 also includes the guide portion 402 for coupling the first and second moldable parts 106 and 108 to the third moldable part 104 and adjusting the first and second moldable parts relative to the third moldable part.

As illustrated in FIGS. 4A, 4B, and 4E, the top surface 450 of the first and second moldable parts 106 and 108 slopes downward from a front portion to a rear portion of the moldable part. As illustrated in FIG. 4E, the height H₁ or vertical thickness of the front portion of the first and second moldable parts 106 and 108 is greater than the height H₂ or vertical thickness of the rear portion due to the angled or sloped top surface 450. This differential in height between the front and rear portions facilitates positioning of the patient's jaw such that there is vertical separation between the maxilla and the mandible when the oral appliance 800 is positioned or installed in the patient's mouth. The angled or sloped top surfaces 450 cause the mandible of the patient to move downward relative to the maxilla when the oral appliance 800 is installed and the patient's teeth are substantially seated within the impressions formed in the moldable parts. Further, the angled or sloped top surfaces 450 cause the mandible of the patient to move forward relative to the maxilla when the oral appliance 800 is installed and the patient's teeth are substantially seated within the impressions formed in the moldable parts.

As illustrated in FIG. 4E, the angle A₇ of the top surface 450 may vary between about 0 degrees and about 10 degrees relative to horizontal. In certain embodiments of the oral appliance 800, the angle A₇ of the top surfaces 450 of the first and second moldable parts 106 and 108 is between about 7 degrees and about 8 degrees; about 8 degrees; or 7.9 degrees relative to horizontal. Further, the height H₁ of the front portion of the first and second moldable parts 106 and 108 may vary between about 0 inch and about 1 inch, the height H₂ of the rear portion may vary between about 0 inch and about 1 inch, and the length L of the first and second moldable parts may vary between about 0 inches and about 3 inches. In certain embodiments, the height H₁ is about 0.38 inch, the height H₂ is about 0.25 inch, and the length L is about 1.37 inches.

As illustrated in FIGS. 5A-5C, the third moldable part 104 includes two base portions 500 and a moldable portion 502. As shown, the moldable portion 502 includes a plurality of spaced protrusions or bosses 506 (i.e., male portion) extending upward from a top surface of the moldable portion. The spaced protrusions 506 are configured to mate with a plurality of spaced openings 508 (i.e., female portion) in the base portions 500 (or vice versa). These protrusions 506 and openings 508 permit the moldable portion 502 to be removably coupled to the base portions 500. However, the base portions 500 and the moldable portion 502 may be made of a single piece of material or a plurality of materials attached together or integrally formed to form the third moldable part 104. Furthermore, the base portions 500 and the moldable portion 502 may be attached together or integrally formed in a variety of ways, such as, for example, laminated, overmolded, or with one or more fasteners. As described in greater detail below, the base portions 500 also includes the track portion 504 for coupling the first and second moldable parts 106 and 108 to the third moldable part 104 and adjusting the first and second moldable parts relative to the third moldable part.

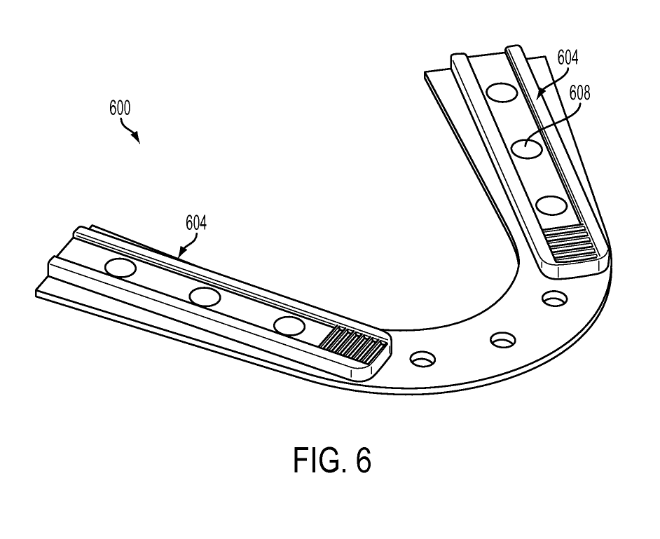

In certain embodiments, the base portion of the third moldable part may comprise a single member having two track portions. For example, FIG. 6 illustrates a base portion 600 according to an embodiment of the present application. The base portion 600 comprises a U-shaped member having two track portions 604 for coupling the first and second moldable parts 106 and 108 to the third moldable part 104 and adjusting the first and second moldable parts relative to the third moldable part. Further, the base portion 600 comprises spaced openings 608 configured to mate with spaced protrusions extending from the moldable portion of the third moldable part 104.

As illustrated in FIGS. 5A and 5B, the third moldable part 104 is generally U-shaped and symmetrical about the centerline 580. As illustrated in FIG. 5B, the centerline 582 of the base portions 500 and track portions 504 may extend at an angle A₈ relative to the centerline 580 of the third moldable part 104. The angle A₈ may vary between about 0 degrees and about 50 degrees. In certain embodiments, the angle A₈ is about 24 degrees.

Further, the interior sidewall 590 and exterior sidewall 592 of the third moldable part 104 may extend at angles A₉ and A₁₀, respectively, relative to the centerline 580. The angle A₉ may vary between about 0 degrees and about 50 degrees and the angle A₁₀ may vary between about 0 degrees and about 50 degrees. In certain embodiments, the angle A₉ is about 21 degrees and the angle A₁₀ is about 27 degrees. The radius R_{I} of the curved portion of the interior sidewall 590 may vary between about 0 inch and about 1 inch and the radius R_{E} of the curved portion of the exterior sidewall 592 may vary between about 0 inch and about 1 inch. In certain embodiments, the radius R_{I} is about 0.52 inch and the radius R_{E} is about 0.92 inch.

As illustrated in FIG. 5C, the height H₁ or vertical thickness of the front or anterior portion of the moldable portion 502 may be greater than the height H₂ or vertical thickness of the rear or posterior portion. The height H₁ of the front portion of the moldable portion 502 may vary between about 0 inch and about 1 inch and the height H₂ of the rear portion may vary between about 0 inch and about 1 inch. In certain embodiments, the height H₁ is about 0.24 inch and the height H₂ is about 0.21 inch.

As illustrated in FIGS. 8A-8C, the guide portions 402 of the first and second moldable parts 106 and 108 are configured to mate with the track portions 504 of the third moldable part 104. The guide portions 402 are configured to slide horizontally along the track portions 504 to couple the first and second moldable parts 106 and 108 to the third moldable part 104. The interior surfaces or sidewalls of the guide portion 402 and the mating exterior surfaces or sidewalls of the track portion 504 may be flared or angled to prohibit the first and second moldable parts 106 and 108 from being removed from the third moldable part 104 (e.g., in a vertical direction). For example, as illustrated in FIGS. 7A and 7B, the guide portion 402 comprises angled surfaces 768 that abut corresponding angled surfaces 776 of the track portion 504 when the first and second moldable parts 106 and 108 are coupled to the third moldable part 104. In other embodiments, however, the track and guide portions may be replaced with any type of mating arrangement capable of coupling the first and second moldable parts 106 and 108 to the third moldable part 104 and/or adjusting the first and second moldable parts relative to the third moldable part, such as, for example, mating arrangements that comprise keys, keyways, notches, grooves, posts, openings, etc. and permit coupling or adjustment of corresponding structures.

Furthermore, as illustrated in FIGS. 8A-8C, the guide portion 402 slides along the track portion 504 to permit horizontal adjustment of the first and second moldable parts 106 and 108 relative to the third moldable part 104. The guide portion 402 also includes a lip or catch 406 that mates with spaced notches or grooves 520 of the track portion 504. The lip 406 seats within the spaced notches 520 to hold the first and second moldable parts 106 and 108 in position relative to the third moldable part 104. As shown, the notches 520 are spaced approximately 1 mm apart to permit 1 mm increments of adjustment of the first and second moldable parts 106 and 108 relative to the third moldable part 104. However, in other embodiments, the track portion 504 may include more or less notches and the notches may be spaced at a variety of increments (e.g., ¼ mm, ½ mm, ¾ mm, 1 ¼ mm, 1 ½ mm, 1 ¾ mm, 2 mm, and so forth) to permit adjustment of the first and second moldable parts 106 and 108 relative to the third moldable part 104. In other embodiments, the third moldable part comprises one or more guide portions that slide along one or more track portions of the first and second moldable parts to permit horizontal adjustment of the first and second moldable parts relative to the third moldable part.

The first and second moldable parts 106 and 108 may be adjusted relative to the third moldable part 104 such that the oral appliance 800 fits a specific patient's measurements. Furthermore, the position of the patient's jaw may be adjusted by moving the first and second moldable parts 106 and 108 relative to the third moldable part 104. For example, moving the first and second moldable parts 106 and 108 horizontally relative to the third moldable part 104 permits the mandible of the patient to be moved more or less forward when the oral appliance 800 is positioned or installed in the patient's mouth and the patient's teeth are substantially seated within the impressions formed in the moldable parts. The oral appliance 800 may be removed from the patient's mouth and re-adjusted at any time.

In certain embodiments, the molding device 100 is configured such that the moldable parts are molded to create an impression of the patient's teeth with the teeth in a first position, such as, for example, normal occlusion. The moldable parts are then coupled together to form the oral appliance 800. The first and second moldable parts 106 and 108 are adjusted relative to the third moldable part 104 such that the oral appliance 800 holds the teeth in the first position. In the first position, the lip 406 of the guide portion 402 is seated within the spaced notches 520 of the track portion 504 at a neutral position. Further, the rear edges of the first and second moldable parts 106 and 108 may be flush or aligned with the rear edge of the third moldable part 104 when the oral appliance 800 is in the first or neutral position. One or both of the first and second moldable parts 106 and 108 may then be adjusted backward or forward relative to the neutral position such that the mandible is moved relative to the first position when the oral appliance 800 is inserted in the patient's mouth.

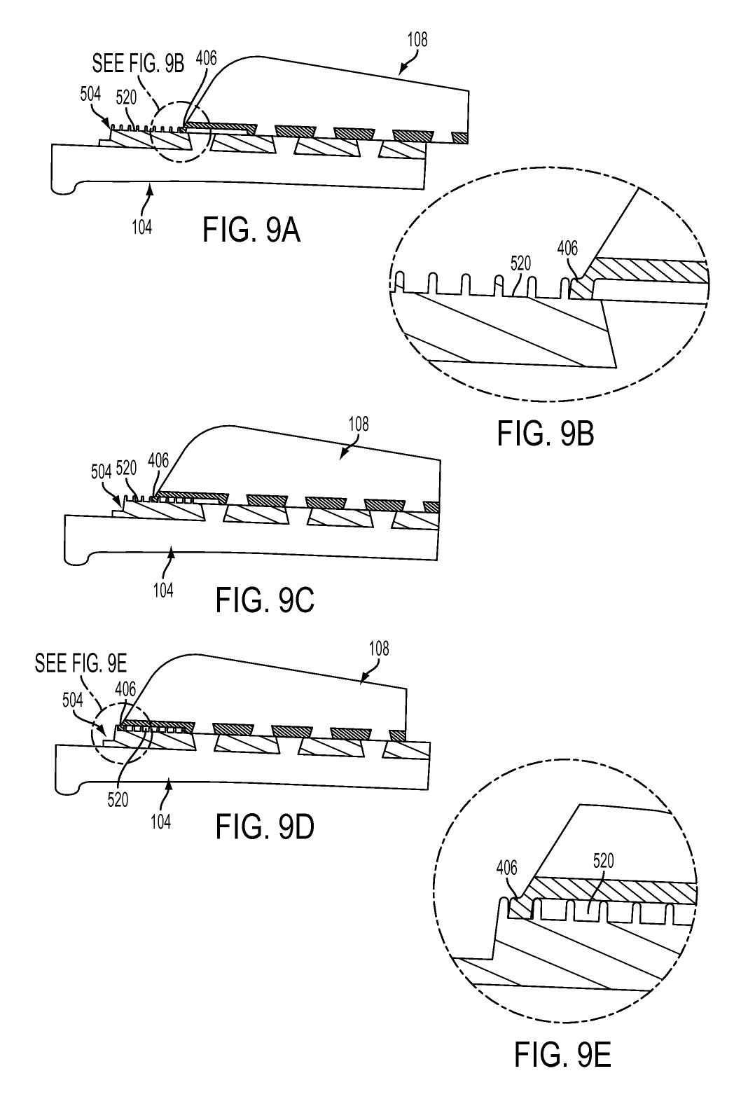

FIGS. 9A-9E illustrate adjustment of the first and second moldable parts 106 and 108 relative to the third moldable part 104 according to an embodiment of the present application. FIG. 9C illustrates the oral appliance 800 in the first or neutral position. In this position, the lip 406 of the guide portion 402 is seated within a middle notch of the track portion 504 and the rear edges of the first and second moldable parts 106 and 108 are flush with the rear edge of the third moldable part 104. FIGS. 9A-9B illustrate the first and second moldable parts 106 and 108 moved fully backward relative to the third moldable part 104, e.g., 5 mm backward, such that the lip 406 is seated within the first or most rearward notch of the track portion 504. FIGS. 9D-9E illustrate the first and second moldable parts 106 and 108 moved fully forward relative to the third moldable part 104, e.g., 3 mm forward, such that the lip 406 is seated within the last or most forward notch of the track portion 504. Moving the first and second moldable parts 106 and 108 backward relative to the third moldable part 104 moves the patient's mandible forward relative to the maxilla when the oral appliance 800 is positioned or installed in the patient's mouth. Conversely, moving the first and second moldable parts 106 and 108 forward relative to the third moldable part 104 moves the patient's mandible backward relative to the maxilla when the oral appliance 800 is positioned or installed in the patient's mouth.

In certain embodiments, the track portion 504 and/or the guide portion 402 may comprise position indicators, such as a hot stamp, impression, marking, or other indication, that identifies the position of the first and second moldable parts 106 and 108 relative to the third moldable part 104. For example, the bottom of the spaced notches 520 may comprise position indicators for the neutral position and the various forward and backward positions relative to the neutral position, e.g., +/−1 mm position indicators.

The base portions 404 and 500 of the moldable parts generally comprise a first material that is different from a second material used for the moldable portions 400 and 502 of the moldable parts. The first material may be more rigid than the second material to facilitate coupling, adjustment, and/or re-adjustment of the moldable parts. Furthermore, a softer second material facilitates creation of the impression of the user's teeth.

The moldable portions 400 and 502 of the moldable parts are also generally made of a softer material than that used for the frame. Furthermore, the material of the moldable portions 400 and 502 does not fuse with the material of the frame after the molding device 100 is placed in hot or boiling water to soften the polymer of the moldable portions (e.g., for approximately 30 seconds, 45 seconds, 1 minute, or longer). As such, the moldable parts may be easily removed from the frame after creation of the impression.

The moldable portions 400 and 502 of the moldable parts may be made of a variety of moldable materials that may be used in the mouth. The moldable portions 400 and 502 may be made of a single piece of material or a plurality of materials attached together or integrally formed to form the moldable portion, such as, for example, laminated, overmolded, or otherwise secured together. In certain embodiments, the moldable portions 400 and 502 of the moldable parts comprise an ethylene vinyl acetate (EVA) copolymer having a 45% vinyl acetate content (e.g., Lanxess Levamelt® 450 described above) and the base portions 404 and 500 of the moldable parts comprise an ethylene vinyl acetate (EVA) copolymer having a 28% vinyl acetate content (e.g., Ateva® 2803G described above). However, a variety of other materials may be used.

An exemplary method of forming the oral appliance 800 includes placing the molding device 100 in boiling water to soften the polymer of the moldable parts (e.g., for approximately 30 seconds, 45 seconds, 1 minute, or longer). The molding device 100 is then removed from the water and inserted into the patient's mouth. The patient then bites down on the moldable parts to create an impression. Once the impression has been made, the molding device 100 is set aside to air dry, allowing the moldable parts to harden and solidifying the impression. Once the moldable parts have dried and hardened, the molding device 100 is disassembled and the frame 102 may be set aside or discarded. The moldable parts—upper right portion 106, upper left portion 108, and the lower portion 104—are assembled to form the oral appliance 800. The oral appliance 800 may be assembled to fit a specific patient's measurements by adjusting the upper portions 106 and 108 relative to the lower portion 104. Further, as described above, the position of the patient's jaw may be adjusted by moving the upper portions 106 and 108 relative to the lower portion 104. The oral appliance 800 may be removed from the patient's mouth and re-adjusted at any time.

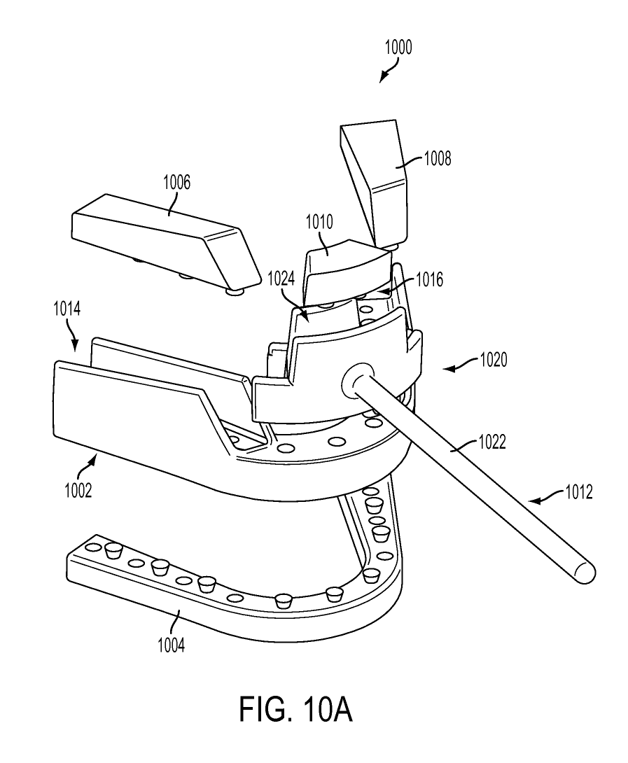

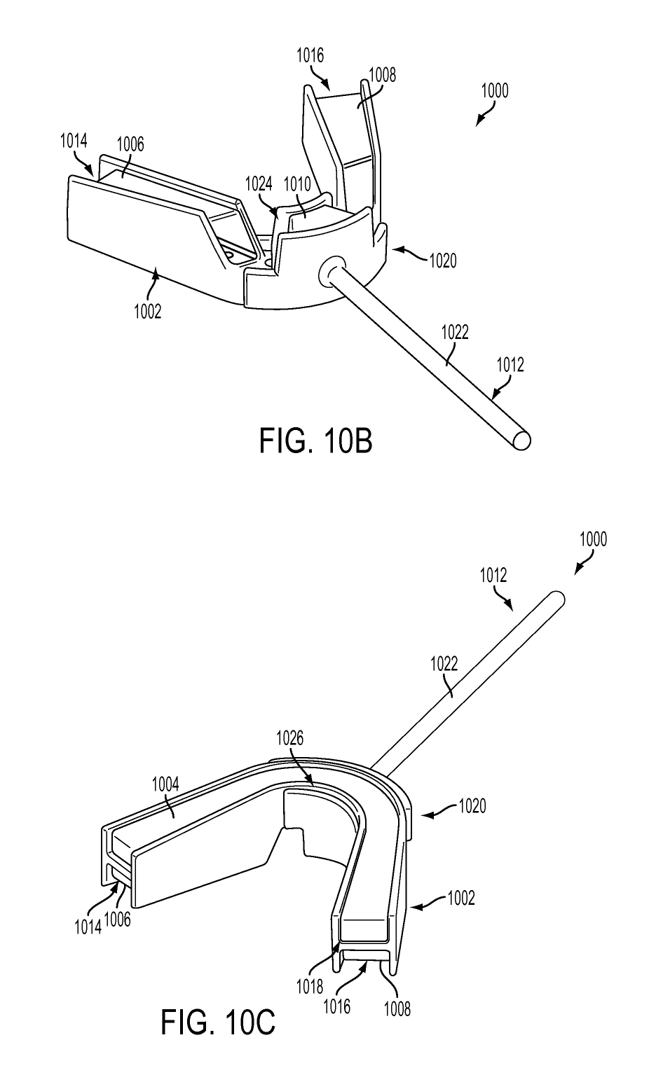

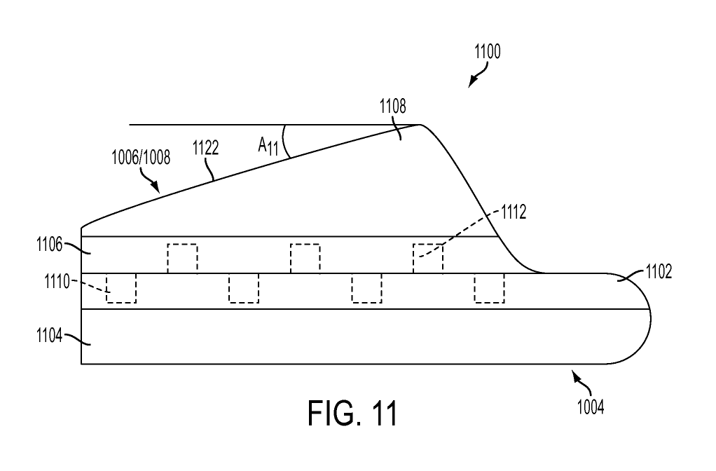

FIGS. 10A-10C disclose a molding device 1000 for making an oral appliance 1100 (FIG. 11) according to an embodiment of the present application. As shown, the molding device 1000 is U-shaped and configured to fit a human mouth. The molding device 1000 comprises a frame 1002, a plurality of moldable parts, and a handle or holder 1012. The moldable parts include three upper portions—a right and left posterior portion 1006 and 1008 and an anterior portion 1010—and a lower portion 1004. Once molded, the upper posterior portions 1006 and 1008 of the moldable parts are removably coupled to the lower portion 1004 to form the oral appliance 1100 of the present application. The upper anterior portion 1010 is generally discarded after molding is complete. However, in some embodiments, the upper anterior portion 1010 may be coupled to the lower portion 1004 in forming the oral appliance.

The frame 1002 of the molding device 1000 acts as the form or mold for the upper posterior portions 1006 and 1008 and the lower portion 1004 of the moldable parts. The frame 1002 comprises sidewalls extending upward and downward from a U-shaped center portion to form upper and lower channels. The moldable parts are disposed within and at least partially fill the upper and lower channels of the frame 1002. The upper and lower channels facilitate molding of the moldable parts by prohibiting the movement and/or outward deformation of the moldable parts as the impression of the upper and lower teeth is formed.

The upper sidewalls and the center piece of the frame 1002 form two upper channels 1014 and 1016. Each upper channel 1014 and 1016 is configured to at least partially surround a portion of the upper posterior teeth (i.e., the molars and bicuspids) of the patient. The right and left posterior portions 1006 and 1008 of the moldable parts at least partially fill the upper channels 1014 and 1016. The shape of the upper channels 1014 and 1016 follow the shape of the upper posterior portions 1006 and 1008 to facilitate molding of these portions.

As illustrated in FIGS. 10A-10C, the two upper channels 1014 and 1016 of the frame 1002 have a varying depth. A top edge of each upper sidewall that forms the upper channels 1014 and 1016 extends downward from a front or leading edge of the sidewall to a rear edge of the sidewall such that the channels are deeper around a front portion of the upper posterior teeth than the rear. The angle of the sloping top edge is generally between about 0 degrees and about 10 degrees relative to horizontal. In certain embodiments, the depth of the upper channel 1014 and 1016 may be no greater than 0.5 inch at the front of the channel and no greater than 0.3 inch at the rear of the channel. Further, the front or leading edge of each upper sidewall extends rearward from the center portion to the top edge at an angle between about 0 degrees and about 45 degrees relative to vertical. In one embodiment of the molding device 1000, the angle of the sloping top edge of each upper sidewall is about 8 degrees relative to horizontal and the angle of the front edge is about 30 degrees relative to vertical.

The lower sidewalls and the center portion of the frame 1002 form a continuous lower channel 1018. The lower channel 1018 is configured to at least partially surround the lower teeth of the patient. The lower portion 1004 of the moldable parts is disposed within and at least partially fills the lower channel 1018. The shape of the lower channel 1018 follows the shape of the lower portion 1004 to facilitate molding of this portion. The lower channel 1018 may be slightly deeper around the lower anterior teeth than the lower posterior teeth. For example, the depth of the lower channel 1018 may be no greater than 0.4 inch at the front or anterior portion of the channel and no greater than 0.3 inch at the rear of the channel.

The upper and lower channels of the frame 1002 include features that are configured to mate with corresponding features on the moldable parts filling the channel. The features on the channel and/or the moldable part may include, for example, rails or tracks, guides, openings, bosses, protrusions, barbs, notches, grooves, ridges, slots, or the like of various shapes and sizes. The features are configured to facilitate alignment of the moldable part in the channel and prohibit movement of the moldable part as the impression of the upper and lower teeth is formed. For example, as illustrated in FIGS. 10A-10C, the center portion of the frame 1002 includes frustro conical shaped openings configured to mate with corresponding frustro conical shaped bosses on the upper posterior portions 1006 and 1008 and the lower portion 1004 of the moldable parts. Further, as described below in reference to FIG. 11, the features on the moldable parts are configured to facilitate attachment and adjustment of the upper posterior portions 1006 and 1008 to the lower portion 1004 when they are assembled to form the oral appliance 1100.

As illustrated in FIGS. 10A-10C, the frame 1002 is made from a single piece of material. However, in other embodiments, the frame 1002 may comprise multiple pieces or components attached together or integrally formed to produce the frame. Further, the frame 1002 may be made of a variety of materials that may be used in the mouth. For example, in one embodiment, the frame 1002 is made of a thermoplastic polymer such as polypropylene. This material facilitates removal of the frame 1002 from the moldable parts after molding is completed. In another embodiment, the frame 1002 is made of an ethylene vinyl acetate (EVA) copolymer. One example of this material is Ateva® 2803G having a 28% vinyl acetate content; a melt index of 3.0 g/10 min; a density of 952 kg/m²; a vicat softening point of 44° C.; and a hardness of 81 (Shore A) and 28 (Shore D). However, a variety of other materials may be used.

As illustrated in FIGS. 10A-10C, the holder 1012 of the molding device 1000 includes an attachment portion 1020 and an elongated member 1022 extending from the attachment portion. The holder 1012 is configured to be removably attached to the frame 1002 for use during the forming process such that the user may be positioned away from the hot or boiling water. Further, the attachment portion 1020 of the holder 1012 acts as the form or mold for the upper anterior portion 1010 of the moldable parts. During the molding process, the upper anterior portion 1010 provides resistance against one or more of the upper anterior teeth (i.e., the incisors and cuspids) when the patient bites down to facilitate formation of the upper and lower teeth impression.

As illustrated in FIGS. 10A-10C, the attachment portion 1020 of the holder 1012 is curved to follow the shape of the anterior portion of the frame 1002. The attachment portion 1020 includes an upper channel 1024 and a lower channel 1026. The lower channel 1026 of the attachment portion 1020 is shaped and sized such that it may be removably coupled to the anterior portion of the frame 1002. As shown, the lower channel 1026 is sized to fit around the center portion and lower sidewalls of the anterior portion of the frame 1002. The lower channel 1026 may be removably coupled to the frame 1002 in a variety of ways, such as, for example, with a friction or interference fit, fastener, clamp, Velcro®, adhesive, or the like. In one embodiment, the lower channel 1026 is shaped and sized such that it may be removably coupled to the anterior portion of the frame 1002 with a friction fit such that it “clamps” around the frame.

The upper channel 1024 of the attachment portion 1020 is configured to at least partially surround one or more of the upper anterior teeth of the patient. The upper anterior portion 1010 of the moldable parts at least partially fills the upper channel 1024. The shape of the upper channel 1024 follows the shape of the upper anterior portion 1010 to facilitate molding of this portion.

As illustrated in FIGS. 10A-10C, the holder 1012 is made from a single piece of material. However, in other embodiments, the holder 1012 may comprise multiple pieces or components attached together or integrally formed to produce the holder. Further, the holder 1012 may be made of a variety of materials that may be used in the mouth. The holder 1012 may be made from the same material as the frame 1002. For example, in one embodiment, the holder 1012 is made of a thermoplastic polymer such as polypropylene. This material facilitates removal of the holder 1012 from the moldable parts and/or the frame 1002 after molding is completed. In another embodiment, the holder 1012 is made of an ethylene vinyl acetate (EVA) copolymer. One example of this material is Ateva® 2803G having a 28% vinyl acetate content; a melt index of 3.0 g/10 min; a density of 952 kg/m²; a vicat softening point of 44° C.; and a hardness of 81 (Shore A) and 28 (Shore D). However, a variety of other materials may be used

Once molded, the upper posterior portions 1006 and 1008 of the moldable parts are removably coupled to the lower portion 1004 to form the oral appliance 1100 shown in FIG. 11. Similar to the oral appliance 800 discussed above, when inserted into the patient's mouth, the oral appliance 1100 is configured to maintain the mandible of the patient in an open and forward position relative to the normal posture of the jaw during sleep to reduce snoring and/or apneic episodes caused by OSA. Positioning the jaw in this manner helps to keep the airway of the patient open during sleep.

As illustrated in FIG. 11, the top surfaces 1122 of the upper posterior portions 1006 and 1008 are angled downward from the front to the rear of the portion. When the oral appliance 1100 is inserted into the patient's mouth, the angled top surfaces 1122 of the upper posterior portions 1006 and 1008 position the jaw such that there is vertical separation between the maxilla and mandible. As illustrated in FIG. 11, the angle A₁₁ of the top surfaces 1122 may be between about 0 degrees and about 10 degrees relative to horizontal. In certain embodiments of the oral appliance 1100, the angle A₁₁ of the top surface 1122 of each upper posterior portion 1006 and 1008 is between about 7 degrees and about 8 degrees; about 8 degrees; or 7.9 degrees relative to horizontal.

Further, the oral appliance 1100 is configured such that the mandible of the patient is positioned forward relative to the maxilla when the oral appliance is inserted into the patient's mouth. Coupling the molded upper posterior portions 1006 and 1008 to the molded lower portion 1004 permits the mandible to be moved forward when the oral appliance 1100 is inserted into the patient's mouth.

In certain embodiments, the upper posterior portions 1006 and 1008 are adjustable relative to the lower portion 1004. As such, the oral appliance may be configured to fit a specific patient's measurements. Further, the position of the patient's jaw may be adjusted by moving the upper posterior portions 1006 and 1008 relative to the lower portion 1004. For example, moving the upper posterior portions 1006 and 1008 horizontally relative to the lower portion 1004 permits the mandible to be moved more or less forward when the oral appliance is inserted in the patient's mouth. Further, moving the upper posterior portions 1006 and 1008 vertically relative to the lower portion 1004 may permit more or less vertical separation between the maxilla and mandible when the oral appliance is inserted in the patient's mouth. The oral appliance may be removed from the patient's mouth and re-adjusted at any time.

The upper posterior portions 1006 and 1008 may be adjusted relative to the lower portion 1004 in a variety of ways. For example, as stated above, the bottom surface of the upper posterior portions 1006 and 1008 and the top surface of the lower portion 1004 may include features such as rails or tracks, guides, openings, bosses, protrusions, barbs, notches, grooves, ridges, slots, or the like of various shapes and sizes. The features on the bottom surface of the upper posterior portions 1006 and 1008 are configured to mate with the features on the top surface of the lower portion 1004 to couple the portions together and permit the portions to be adjusted relative to one another.

For example, as illustrated in FIG. 11, spaced protrusions or bosses 1110 (i.e., male portion) extend downward from the bottom of the upper posterior portions 1006 and 1008 and are configured to mate with spaced openings (i.e., female portion) in the top of the lower portion 1004. Further, spaced protrusions or bosses 1112 extend upward from the top of the lower portion 1004 and are configured to mate with spaced openings in the bottom of the upper posterior portions 1006 and 1008. These protrusions 1110 and 1112 and openings permit the upper posterior portions 1006 and 1008 to be removably coupled to the lower portion 1004 at one of a plurality of positions or settings and permit adjustment of the upper posterior portions relative to the lower portion.

The moldable parts are generally made of a softer material than that used for the frame 1002 and/or holder 1012. The moldable parts may be made of a variety of moldable materials that may be used in the mouth. Further, the moldable parts may be made of a single piece of material or a plurality of materials attached together or integrally formed to form the moldable part, such as, for example, laminated, overmolded, or otherwise secured together. For example, in certain embodiments, the bottom surface (or lower part) of the upper posterior portions 1006 and 1008 and the top surface (or upper part) of the lower portion 1004 comprise a first material that is more rigid or sturdier than a second material used for the upper part of the upper posterior portions and the lower part of the lower portion. As such, the more rigid first material facilitates coupling and adjustment of the upper posterior portions 1006 and 1008 and the lower portion 1004, such as with the features described above, and the softer second material facilitates creation of the impression of the user's teeth.

For example, as illustrated in FIG. 11, the lower part 1106 of the upper posterior portions 1006 and 1008 and the upper part 1102 of the lower portion 1004 comprise a first material that is different from a second material used for the upper part 1108 of the upper posterior portions and the lower part 1104 of the lower portion. As shown, the protrusions 1110 and 1112 and openings are made of the first material. The first material may be more rigid than the second material to facilitate coupling, adjustment, and/or re-adjustment of the upper posterior portions 1006 and 1008 and the lower portion 1004. Further, a softer second material facilitates creation of the impression of the user's teeth.

In certain embodiments, the moldable parts are made from one or more ethylene vinyl acetate (EVA) copolymer. For example, in certain embodiments, the lower part 1106 of the upper posterior portions 1006 and 1008 and the upper part 1102 of the lower portion 1004 may comprise an EVA copolymer having a 28% vinyl acetate content. One example of this material is Ateva® 2803G having a 28% vinyl acetate content; a melt index of 3.0 g/10 min; a density of 952 kg/m²; a vicat softening point of 44° C.; and a hardness of 81 (Shore A) and 28 (Shore D). Further, in certain embodiments, the upper part 1108 of the upper posterior portions 1006 and 1008 and the lower part 1104 of the lower portion 1004 comprise an ethylene vinyl acetate (EVA) copolymer having a 45% vinyl acetate content. One example of this material is Lanxess Levamelt® 450 having a 45±1.5% vinyl acetate content; a melt flow rate at 190° C./21.2 N of 3±2 g/10 min; and a specific gravity of approximately 0.99. However, a variety of other materials may be used.

An exemplary method of forming the oral appliance 1100 of the present application includes placing the molding device 1000 in boiling water for approximately 30 seconds to soften the polymer of the moldable parts. The molding device 1000 is then removed from the water and inserted into the patient's mouth. The patient then bites down on the moldable parts to create an impression. Once the impression has been made, the molding device 1000 is set aside to air dry, allowing the moldable parts to harden and solidifying the impression. Once the moldable parts have dried and hardened, the molding device 1000 is disassembled and the frame 1002 and holder 1012 may be set aside or discarded. Further, the upper anterior portion 1010 of the moldable parts may be set aside or discarded. The remaining three moldable parts—upper right posterior portion 1006, upper left posterior portion 1008, and the lower portion 1004—are assembled to form the oral appliance 1100. The oral appliance 1100 may be assembled to fit a specific patient's measurements by adjusting the upper posterior portions 1006 and 1008 relative to the lower portion 1004. Further, as described above, the position of the patient's jaw may be adjusted by moving the upper posterior portions 1006 and 1008 relative to the lower portion 1004. The oral appliance 1100 may be removed from the patient's mouth and re-adjusted at any time.

As described herein, when one or more components are described as being connected, joined, affixed, coupled, attached, or otherwise interconnected, such interconnection may be direct as between the components or may be in direct such as through the use of one or more intermediary components. Also as described herein, reference to a “member,” “component,” or “portion” shall not be limited to a single structural member, component, or element but can include an assembly of components, members or elements.

While the present invention has been illustrated by the description of embodiments thereof, and while the embodiments have been described in considerable detail, it is not the intention of the applicants to restrict or in any way limit the scope of the invention to such details. Additional advantages and modifications will readily appear to those skilled in the art. For example, where components are releasably or removably connected or attached together, any type of releasable connection may be suitable including for example, locking connections, fastened connections, tongue and groove connections, etc. Still further, component geometries, shapes, and dimensions can be modified without changing the overall role or function of the components. Therefore, the inventive concept, in its broader aspects, is not limited to the specific details, the representative apparatus, and illustrative examples shown and described. Accordingly, departures may be made from such details without departing from the spirit or scope of the applicant's general inventive concept.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, devices and components, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

We claim:

1. An oral appliance, comprising:
a first moldable part and a second moldable part, wherein each of the first and second moldable parts is configured to form an impression of one or more of a user's upper teeth, and wherein each of the first and second moldable parts comprise a front portion, a rear portion, and a top surface sloping downward from the front portion to the rear portion; and
a third moldable part configured to form an impression of one or more of the user's lower teeth, wherein the first and second moldable parts are coupled to the third moldable part to form the oral appliance, and wherein the first and second moldable parts are adjustable relative to the third moldable part; and
wherein the top surfaces of the first and second moldable parts facilitate positioning the user's jaw such that there is vertical separation between the maxilla and the mandible when the oral appliance is installed in the user's mouth.

2. The oral appliance of claim 1, wherein the oral appliance is configured such that the mandible of the user is positioned forward relative to the maxilla when the oral appliance is installed in the user's mouth.

3. The oral appliance of claim 1, wherein the angle between the top surfaces of the first and second moldable parts and the horizontal is between about 1 degree and about 10 degrees.

4. The oral appliance of claim 1, wherein the angle between the top surfaces of the first and second moldable parts and the horizontal is between about 7 degrees and about 8 degrees.

5. The oral appliance of claim 1, wherein the first and second moldable parts are independently adjustable relative to the third moldable part.

6. The oral appliance of claim 1 further comprising one or more adjustment devices for adjusting the first and second moldable parts relative to the third moldable part.

7. The oral appliance of claim 6, wherein the adjustment device comprises one or more guide portions and one or more track portions, and wherein the guide portion is configured to mate with the track portion such that the guide portion is movable relative to the track portion.

8. The oral appliance of claim 7, wherein the first moldable part comprises a first guide portion, the second moldable part comprises a second guide portion, and the third moldable part comprises a first track portion and a second track portion, and wherein the first guide portion is configured to mate with the first track portion such that the first moldable part is movable relative to the third moldable part, and wherein the second guide portion is configured to mate with the second track portion such that the second moldable part is movable relative to the third moldable part.

9. The oral appliance of claim 7, wherein the track portion comprises one or more openings configured to receive a catch of the guide portion to hold the guide portion in position relative to the track portion.

10. The oral appliance of claim 9, wherein the track portion comprises a plurality of openings spaced at least partially along the length of the track portion to permit selective adjustment of the guide portion relative to the track portion.

11. The oral appliance of claim 1, wherein the first, second, and third moldable parts comprise an ethylene vinyl acetate copolymer.

12. The oral appliance of claim 1, wherein the first moldable part is interchangeable with the second moldable part.

13. A device for making an oral appliance, the device comprising:
a first moldable part and a second moldable part, wherein each of the first and second moldable parts is configured to form an impression of one or more of a user's upper teeth;
a third moldable part configured to form an impression of one or more of the user's lower teeth; and
a frame for molding the first, second, and third moldable parts, wherein the frame comprises a first upper channel, a second upper channel, and one or more lower channels, and wherein the first moldable part is at least partially disposed within the first upper channel, the second moldable part is at least partially disposed within the second upper channel, and the third moldable part is at least partially disposed within the one or more lower channels.

14. The device of claim 13, wherein the frame comprises a first lower channel and a second lower channel, and wherein the third moldable part is at least partially disposed within the first and second lower channels.

15. The device of claim 14, wherein the frame comprises a first center portion, a second center portion, a first upper sidewall extending upward from the first center portion to at least partially form the first upper channel, a first lower sidewall extending downward from the first center portion to at least partially form the first lower channel, a second upper sidewall extending upward from the second center portion to at least partially form the second upper channel, and a second lower sidewall extending downward from the second center portion to at least partially form the second lower channel.

16. The device of claim 13, wherein the frame comprises one or more center portions, a plurality of first sidewalls extending upward from the one or more center portions to form the first and second upper channels, and a plurality of second sidewalls extending downward from the one or more center portions to form the one or more lower channels.

17. The device of claim 13 further comprising a holder extending from the frame.

18. The device of claim 13, wherein the first and second channels comprise a front portion and a rear portion, and wherein the depth of the first and second channels varies between the front portion and the rear portion of the channel.

19. The device of claim 13, wherein the first and second moldable parts comprise a guide portion configured to mate with a track portion of the first and second upper channels.

20. The device of claim 13, wherein the moldable parts are shaped and configured such that they are frictionally held within the channels of the frame.

21. The device of claim 13, wherein the moldable parts are removable from the frame after molding.

22. The device of claim 13, wherein the frame is made of a different material than the moldable parts to facilitate removal of the moldable parts from the frame after molding.

23. The device of claim 1, wherein the frame comprises right and left portions connected together by an attachment portion, and wherein the attachment portion permits relative movement of the right and left portions of the frame.

24. The device of claim 23, wherein the attachment portion comprises a member having a first end attached to the right portion and a second end attached to the left portion.

25. A method of making an oral appliance, comprising the steps of:
placing a molding device in boiling water, wherein the molding device comprises a first moldable part, a second moldable part, a third moldable part, and a frame, and wherein the moldable parts are disposed within channels of the frame;
removing the molding device from the boiling water;
inserting the molding device in a user's mouth and biting down on the moldable parts to form an impression of the user's teeth in the moldable parts;
removing the molding device from the user's mouth;
removing the moldable parts from the frame of the molding device;
coupling the first and second moldable parts to the third moldable part to form the oral appliance.

26. The method of claim 25, wherein the first and second moldable parts are adjustably coupled to the third moldable part to form the oral appliance, the method further comprising adjusting the first and second moldable parts relative to the third moldable part.

27. The method of claim 26, wherein the impression of the user's teeth is formed with the user's jaw in a first position, the method further comprising adjusting the first and second moldable parts relative to the third moldable part to move the mandible of the user's jaw from the first position to a second position when the oral appliance is installed in the user's mouth.

28. The method of claim 25 further comprising installing the oral appliance in the user's mouth.

29. The method of claim 28, wherein each of the first and second moldable parts comprise a front portion, a rear portion, and a top surface sloping downward from the front portion to the rear portion, and wherein the top surfaces of the first and second moldable parts facilitate positioning the user's jaw such that there is vertical separation between the maxilla and the mandible when the oral appliance is installed in the user's mouth.

30. The method of claim 28, wherein the oral appliance is configured such that the mandible of the user is positioned forward relative to the maxilla when the oral appliance is installed in the user's mouth.
